(19) United States
(12) Patent Application Publication
(10) Pub. No.: US 2022/0095888 A1
(43) Pub. Date: Mar. 31, 2022

(54) MEDICAL SYSTEMS, DEVICES, AND RELATED METHODS

(71) Applicant: Boston Scientific Limited, Galway (IE)

(72) Inventors: Deepak Kumar SHARMA, Muzaffarnafar (IN); Nabarun BHOWMICK, Kolkata (IN); James SCUTTI, Norwell, MA (US)

(73) Assignee: Boston Scientific Limited, Galway (IE)

(21) Appl. No.: 17/486,730

(22) Filed: Sep. 27, 2021

Related U.S. Application Data
(60) Provisional application No. 63/084,103, filed on Sep. 28, 2020.

(51) Int. Cl. A61B 1/008; A61B 1/01; A61B 1/005

(52) U.S. Cl. CPC A61B 1/008; A61B 1/0057; A61B 1/0052; A61B 1/01

(57) ABSTRACT

In one example, a medical device may include a main shaft extending from a proximal end to a distal end; a first articulation shaft coupled to the distal end of the main shaft; a second articulation shaft coupled to a distal end of the first articulation shaft; a first articulation wire coupled proximate to a distal portion of the first articulation shaft and extending longitudinally through the main shaft and the first articulation shaft; and a second articulation wire coupled proximate to a distal portion of the second articulation shaft and extending longitudinally through the main shaft, the first articulation shaft, and the second articulation shaft.

### CROSS-REFERENCE TO RELATED APPLICATIONS

This application claims the benefit of priority from U.S. Provisional Application No. 63/084,103, filed Sep. 28, 2020, which is incorporated by reference herein in its entirety.

### TECHNICAL FIELD

This disclosure generally relates to medical systems, devices, and related methods that may be used to treat a subject. Aspects of the disclosure relate to medical systems, devices, and methods for endoscopic medical procedures, such as articulating one or more medical devices during endoscopic procedures, among other aspects.

### BACKGROUND

Organ walls are composed of several layers: the mucosa (the surface layer), the submucosa, the muscularis (muscle layer), and the serosa (connective tissue layer). In gastrointestinal, colonic, and esophageal cancer, lesions or cancerous masses may form along the mucosa and often extend into the lumens of the organs. Conventionally, the condition is treated by cutting out a portion of the affected organ wall. This procedure, however, may cause discomfort to patients, and pose health risks.

Physicians have adopted minimally invasive techniques, such as endoscopic procedures like endoscopic mucosal resection (EMR) and endoscopic submucosal dissection (ESD). EMR methods are typically used for removal of small cancerous or abnormal tissues (e.g., polyps), and ESD methods are typically used for en bloc removal of large cancerous or abnormal tissues (e.g., lesions). These procedures are generally performed with an endoscope, which is a long, narrow member optionally equipped with a light, imaging equipment, and other instruments. During these procedures, the endoscope may be passed through a percutaneous incision, passed down the throat, or guided through the rectum to reach tissue targeted for resection or dissection, such a tissue having an abnormality such as a lesion or cancerous mass in an affected organ. The lesion is generally identified and marked. The mucosal layer containing the lesion is then separated from the underlying tissue layers using a medical instrument extending through a working channel of the endoscope. The lesion is subsequently removed using the same or different medical instrument. Conventionally, tissue is removed by employing a cutting device such as a wire loop or knife, which may be adapted for electrocautery. Subsequently, excised tissue may be extracted for examination or disposal using a tissue removal device such as a grasper or other device for holding tissue. Both the cutting device and tissue removal device often need to apply a large traction force or lifting force on the tissue during the procedure, and such a lifting force may be limited by conventional devices employing a total of three or five articulating wires which only unitize a single articulating wire at a time to apply the lifting force. The limited lifting force in conventional tissue cutting and grasping devices may cause increased procedure time, patient injury, or other procedural complications.

The systems, devices, and methods of this disclosure may rectify some of the deficiencies described above or address other aspects of the art.

### SUMMARY

Examples of the disclosure relate to, among other things, systems, devices, and methods for performing one or more medical procedures with the medical systems and devices. Each of the examples disclosed herein may include one or more of the features described in connection with any of the other disclosed examples.

In one example, a medical device may include a main shaft extending from a proximal end to a distal end; a first articulation shaft coupled to the distal end of the main shaft; a second articulation shaft coupled to a distal end of the first articulation shaft; a first articulation wire coupled proximate to a distal portion of the first articulation shaft and extending longitudinally through the main shaft and the first articulation shaft; and a second articulation wire coupled proximate to a distal portion of the second articulation shaft and extending longitudinally through the main shaft, the first articulation shaft, and the second articulation shaft.

In other aspects, a medical device may include one or more of the following features. The first articulation shaft may be configured to bend independent of bending of the second articulation shaft, via movement of the first articulation wire; and the second articulation shaft may be configured to bend independently of bending of the first articulation shaft, via movement of the second articulation wire. A tool may be coupled to a distal end of the second articulation shaft, and an actuation wire may extend through the main shaft, the first articulation shaft, and the second articulation shaft, to actuate the tool. A first articulation ring may be coupled to a distal end of the first articulation shaft and may be coupled to a proximal end of the second articulation shaft, and the first articulation wire may be fixedly coupled to the first articulation ring. A second articulation ring may be coupled to a distal end of the second articulation shaft, and the second articulation wire may be fixedly coupled to the second articulation ring. A third articulation wire may extend longitudinally through the main shaft and the first articulation shaft and may be fixedly coupled to the first articulation ring; a fourth articulation wire extending longitudinally through the main shaft, the first articulation shaft, and the second articulation shaft, and the fourth articulation wire may be fixedly coupled to the second articulation ring; a tool may be coupled to a distal portion of the second articulation ring; and an actuation wire may extend longitudinally through the main shaft, the first articulation shaft, the first articulation ring, the second articulation shaft, and the second articulation ring, and the actuation wire may be configured to actuate the tool when moved proximally or distally.

In other aspects, a medical device may include one or more of the following features. A handle may be coupled to the proximal end of the main shaft; and the handle may include a first actuator rotatably coupled to the handle, and movement of the first actuator may move the first articulation wire; and a second actuator coupled to the handle, the second articulation wire, and the main shaft, and the second actuator may be rotatable relative to the main shaft, and pivoting the handle relative to the main shaft may move the second articulation wire. The handle may further include a third actuator configured to actuate the tool. A first connector may be configured to fixedly couple to a working channel port of an endoscope at a distal end of the first connector, and the handle may be configured to rotatably couple to a proximal end of the first connector; and the first connector may be configured to space the handle from a handle of the endoscope. The second actuator may include a ring portion, and the second articulation wire may be coupled to the ring portion; the first articulation wire may extend through a central lumen of the ring portion; and the handle may pivot about a center of the ring portion to move the second articulation wire. Third, fourth and fifth articulation wires may be coupled proximate to a distal portion of the first articulation shaft and may extend longitudinally through the main shaft and the first articulation shaft, and the third, fourth and fifth articulation wires may be coupled to the first actuator; and sixth, seventh, and eighth articulation wires may be coupled proximate to a distal portion of the second articulation shaft and may extend longitudinally through the main shaft, the first articulation shaft, and the second articulation shaft, and the sixth, seventh, and eighth articulation wires may be coupled to the second actuator. The first actuator may include a joystick and a ball, and each of the first, third, fourth, and fifth articulation wires may be coupled to the ball. The first articulation wire may be positioned within a first lumen of the main shaft and the first articulation shaft, the first lumen being directly adjacent to lumens of the main shaft and the first articulation shaft in which the second and sixth articulation wires are positioned; and the fourth articulation wire may be positioned within a second lumen of the main shaft and the first articulation shaft, the second lumen being directly adjacent to lumens of the main shaft and the first articulation shaft in which the seventh and eighth articulation wires are positioned. The handle may be L-shaped; the main shaft may extend distally from a distal end of the handle; the first actuator may extend outward from an exterior surface of the handle and may include a joystick; and the second actuator may be positioned at a distal portion of the handle. Pivoting the joystick may be configured to bend the first articulation shaft; and pivoting the handle relative to the main shaft may be configured to bend the second articulation shaft.

In another example, a medical device may include a main shaft extending from a proximal end to a distal end; a first articulation shaft coupled to the distal end of the main shaft; a second articulation shaft coupled to a distal end of the first articulation shaft; and a handle coupled to the proximal end of the main shaft. The handle may include a first actuator rotatably coupled to the handle, and movement of the first actuator may bend the first articulation shaft independent of bending the second articulation shaft; and a second actuator coupled to the handle, wherein the second actuator may be rotatable relative to the main shaft, and pivoting the handle relative to the main shaft may bend the second articulation shaft independent of bending of the first articulation shaft.

In other aspects, a medical device may include one or more of the following features. The handle may further include a third actuator configured to actuate a tool at a distal portion of the medical device. The handle may be a first handle, and the medical device may also include a first connector configured to fixedly couple to a working channel port of an endoscope at a distal end of the first connector, wherein the first handle is configured to rotatably couple to a proximal end of the first connector; and wherein the first connector is configured to space the handle from a second handle of the endoscope.

In other aspects, a medical device may include a main shaft extending from a proximal end to a distal end; a first articulation shaft coupled to the distal end of the main shaft; a second articulation shaft coupled to a distal end of the first articulation shaft; and a handle coupled to the main shaft, the handle comprising a first actuator and a second actuator. The first articulation shaft may be configured to bend independent of bending of the second articulation shaft upon actuation of the first actuator; and the second articulation shaft may be configured to bend independent of bending of the first articulation shaft upon actuation of the second actuator.

In other aspects, a medical device may include one or more of the following features. The first articulation shaft may include a plurality of lumens, and the second articulation shaft may include a plurality of lumens; and the first articulation shaft may include at least one more lumen than the second articulation shaft.

It may be understood that both the foregoing general description and the following detailed description are exemplary and explanatory only and are not restrictive of this disclosure, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 illustrates a perspective view of a medical device system, according to aspects of this disclosure.

FIG. 2 illustrates a side view of an exemplary medical device, according to aspects of this disclosure.

FIG. 3 illustrates a side view of a distal portion of the exemplary medical device in FIG. 2, according to aspects of this disclosure.

FIG. 4 illustrates a side view of components of the medical device of FIG. 2, according to aspects of the disclosure.

FIG. 5 illustrates a perspective view of components of the medical device of FIG. 2, according to aspects of the disclosure.

FIG. 6 illustrates a portion of the medical device of FIG. 2 cut through axis A, according to aspects of the disclosure.

FIG. 7 illustrates a portion of the medical device of FIG. 2 cut through axis B, according to aspects of the disclosure.

FIG. 8 illustrates a side view of components of the medical device of FIG. 2, according to aspects of the disclosure.

FIG. 9 illustrates a side view of components of the medical device of FIG. 2, according to aspects of the disclosure.

FIG. 10 illustrates a perspective view of components of the medical device of FIG. 2, according to aspects of the disclosure.

FIG. 11 illustrates a side view of a component of the medical device of FIG. 2, according to aspects of the disclosure.

FIGS. 12 and 13 illustrate side views of a distal portion of the medical device of FIG. 2, according to aspects of the disclosure.

FIG. 14 illustrates a perspective view of a component of the medical device of FIG. 2, according to aspects of the disclosure.

FIGS. 15A, 15B, 16A, and 16B illustrate portions of the medical device system of FIG. 1, according to aspects of the disclosure.

FIGS. 17A, 17B, and 17C illustrate components of an articulation section of a medical device, according to aspects of the disclosure.

FIG. 18 illustrates a perspective view of a distal portion of a shaft of the medical device of FIG. 2, according to aspects of the disclosure.

FIG. 19 illustrates a perspective view of a portion of a component of the shaft of FIG. 18, according to aspects of the disclosure.

### DETAILED DESCRIPTION

The terms “proximal” and “distal” are used herein to refer to the relative positions of the components of an exemplary medical system and exemplary medical devices. When used herein, “proximal” refers to a position relatively closer to the exterior of the body or closer to a medical professional using the medical system or medical device. In contrast, “distal” refers to a position relatively further away from the medical professional using the medical system or medical device, or closer to the interior of the body. Proximal and distal directions are labeled with arrows marked “P” and “D”, respectively, throughout the figures. As used herein, the terms “comprises,” “comprising,” “having,” “including,” or other variations thereof, are intended to cover a non-exclusive inclusion, such that a system, device, or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

Embodiments of this disclosure include devices, systems, and methods for manipulating, cutting, grabbing, and/or otherwise treating tissue. In some examples the devices, systems and/or methods discussed herein may be utilized during endoscopic mucosal resection (EMR) and/or endoscopic submucosal dissection (ESD) procedures. In examples, ESD includes endoluminal placement of a grasping device and a cutting device proximate to a target area within the body of a patient. Placement of the grasping and cutting devices may be via a catheter, scope (endoscope, bronchoscope, colonoscope, gastroscope, duodenoscope, etc.), tube, or sheath, inserted into the GI tract via a natural orifice or incision. The orifice can be, for example, the nose, mouth, or anus, and the placement can be in any portion of the GI tract, including the esophagus, stomach, duodenum, large intestine, or small intestine. Placement also can be in other organs reachable via the GI tract. The patient's tissue may be grasped using the grasper and lifted with the grasper, and then the tissue may be cut by the cutting device for subsequent removal from the patient's body.

Reference will now be made in detail to examples of this disclosure described above and illustrated in the accompanying drawings. Wherever possible, the same reference numbers will be used throughout the drawings to refer to the same or like parts.

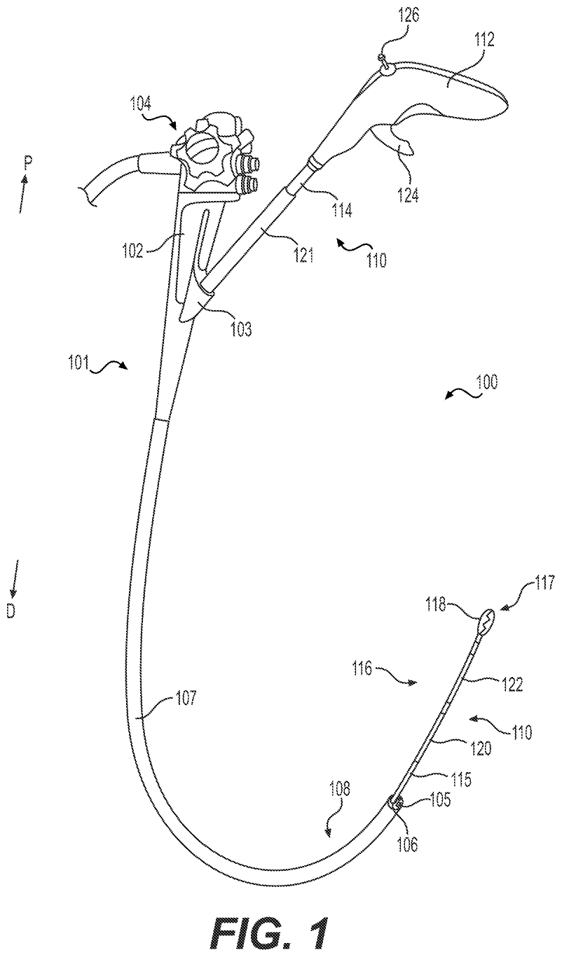

FIG. 1 illustrates a perspective view of an exemplary medical device assembly 100 including an endoscope 101 and a medical device 110. Although medical device assembly 100 is shown with endoscope 101, any other similar insertion device may be used in medical device assembly 100, such as a bronchoscope, colonoscope, gastroscope, duodenoscope, etc. Endoscope 101 may include a handle 102, actuators 104, and a body 107 extending from handle 102 to a distal end 108. A working channel 106 may extend from a working channel port 103 positioned on the handle 102 to an opening at distal end 108. Distal end 108 of endoscope 101 may also include a camera 105, and movement of distal end 108 and functionality of camera 105 may be controlled via one or more actuators 104 on handle 102. Medical device 110 may be configured to be inserted into working channel 106 of endoscope 101.

Medical device 110 may include a handle 112, a main shaft 115 extending from the handle 112 to a distal portion 116 of medical device 110, and a grasper 118 at a distal end 117 of the device. Handle 112 may include a joystick actuator 126, a trigger actuator 124, and proximal connector 114 configured to couple to a distal connector 121. Distal connector 121 may be directly coupled to working channel port 103 of endoscope 101. Main shaft 115 may extend from handle 112 through proximal connector 114 and distal connector 121 into working channel 106 of endoscope, and may be movable within working channel 106. Main shaft 115 may extend longitudinally a length larger than the longitudinal length of endoscope 101, such that once proximal connector 114 is coupled to distal connector 121 and main shaft 115 is pushed through working channel 106, a portion of main shaft 115 extends distally from a distal opening of working channel 106 (shown in FIG. 1). Distal portion 116 of medical device 110 may include a first articulation shaft 120, a second articulation shaft 122, and a grasper 118 at a distal end 117 of medical device 110.

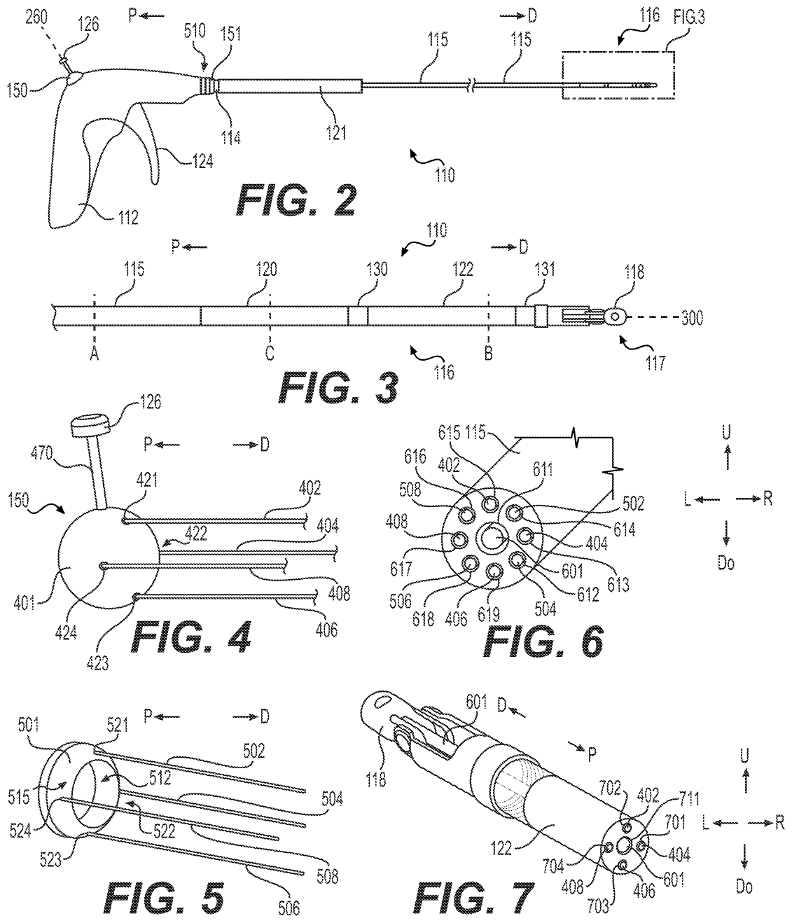

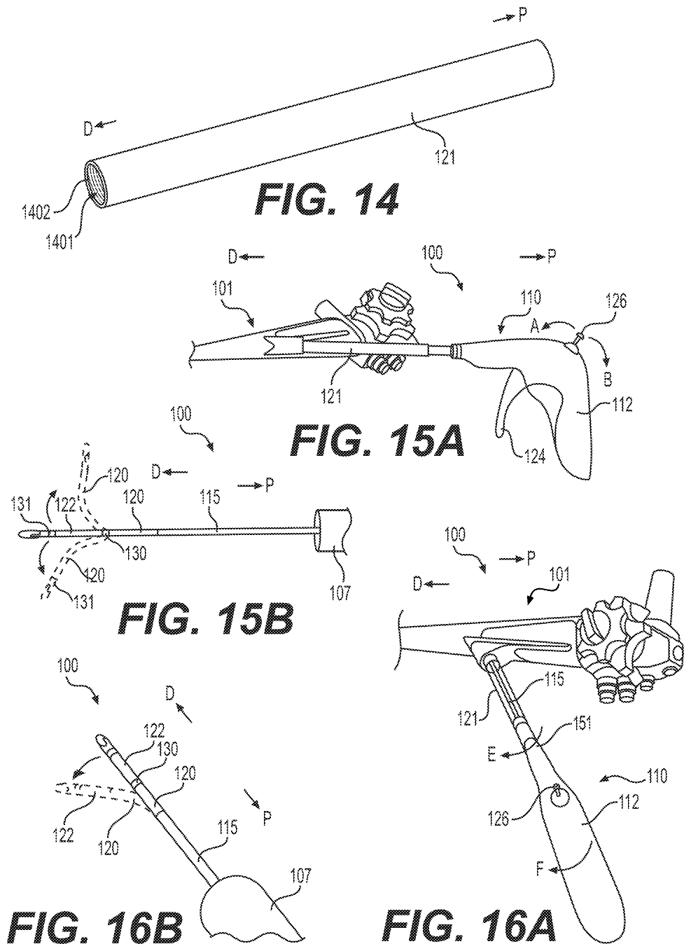

FIG. 2 illustrates a side view of medical device 110 from FIG. 1 removed from endoscope 101. As shown in FIG. 2, handle 112 may be L-shaped and may include trigger actuator 124 extending from a central portion of handle 112. Trigger actuator 124 may be configured to open and close grasper 118 by pulling on trigger actuator 124. In some examples, trigger actuator 124 may be spring-biased towards a closed position, or biased towards a position where grasper 118 is in a closed position. Joystick actuator 126 may extend outward from an exterior surface of handle 112, and may be coupled to handle 112 via a ball joint coupler 150. Joystick actuator 126 may pivot with ball joint coupler 150, relative to handle 112, and may be configured to be actuated via a user's thumb or other fingers. Handle 112 may be coupled to proximal connector 114 via a second ball joint coupler 151. Handle 112 may pivot relative to proximal connector 114 at second ball joint coupler 151. Proximal connector 114 may be configured to rigidly couple to distal connector 121 via insertion into a lumen of distal connector 121. Main shaft 115 may extend from handle 112 and second ball joint coupler 151, through proximal connector 114 and distal connector 121 to distal portion 116 of medical device 110. Main shaft 115 may have a diameter about a central longitudinal axis of main shaft 115 that is less than 2.8 mm, and may be configured to move through a 2.8 mm working channel of a medical device, such as working channel 105 of endoscope 101. To position medical device 110 within working channel 105, a user may first couple distal connector 121 to working channel port 103, such as by screwing threads 1401 (shown in FIG. 14) into corresponding threads at working channel port 103 to fixedly couple distal connector 121 to handle 102 of endoscope 101. A user may then move distal portion 116 of medical device 110 through distal connector 121 and into working channel 105 until proximal coupler 114 is coupled to the proximal end of distal connector 121. In some examples, proximal coupler 114 may allow rotational and translational movement of medical device 110 relative to distal connector 121, such that medical device 110 may be moved proximally, distally, or rotated about its central longitudinal axis 300 within working channel 105.

FIG. 3 shows a side view of distal portion 116 of medical device 110 including main shaft 115, first articulation shaft 120, a first articulation ring 130, second articulation shaft 122, a second articulation ring 131, and grasper 118 at a distal end 117 of medical device 110. In some examples, each of main shaft 115, first articulation shaft 120, first articulation ring 130, second articulation shaft 122, and second articulation ring 131 may be cylindrical and may be configured to move through working channel 106. In some examples, each of main shaft 115, first articulation shaft 120, first articulation ring 130, second articulation shaft 122, and second articulation ring 131 may have approximately the same diameter and circumference about central longitudinal axis 300. One or more of first articulation shaft 120 and second articulation shaft 122 may be made of polyether block amide (PEBAX®) and/or polytetrafluoroethylene (PTFE). In some examples, one or more of first articulation shaft 120 and second articulation shaft 122 may be made of a braided layer and a reflow layer, such as a reflow layer of PEBAX®, applied over the braided layer, of metal or polymer wire. One or more articulation wires 402, 404, 406, 408, 502, 504, 506, 508 may extend through each of main shaft 115, first articulation shaft 120, first articulation ring 130, second articulation shaft 122, and second articulation ring 131. Each of first articulation shaft 120 and second articulation shaft 122 may be configured to bend, twist, and/or otherwise move independently of each other.

First articulation ring 130 may couple first articulation shaft 120 to second articulation shaft 122, and one or more articulation wires 402, 404, 406, 408, 502, 504, 506, 508 may be fixedly coupled to first articulation ring 130. Second articulation ring 131 may couple second articulation shaft 122 to grasper 118, and one or more articulation wires 402, 404, 406, 408, 502, 504, 506, 508 may be fixedly coupled to second articulation ring 131. Articulation rings 130, 131 may be made of a metal or polymer, or any other suitable material. Grasper 118 may be fixedly coupled to second articulation ring 131. In other examples, grasper 118 may be a cutting device such as a knife or scissors assembly, may be an electrode or other electrical cutting tool, may be a water or air jet, may be an suction device, or any other medical tool known in the art. Distal portion 116 of medical device 110 is shown in a neutral position aligned with central longitudinal axis 300 in FIG. 3.

FIG. 4 shows first ball joint coupler 150 and joystick actuator 126 removed from handle 112. As shown in FIG. 4, ball 401 of first ball joint coupler 150 may include four lumens 421, 422, 423, 424 configured to receive four articulation wires 402, 404, 406, 408. Each articulation wire 402, 404, 406, 408 may be fixedly coupled to ball 401, such that pivoting joystick actuator 126 and ball joint coupler 150 relative to a remainder of handle 112 moves at least two of articulation wires 402, 404, 406, 408. Joystick actuator 126 may be moved by a user's thumb or other fingers to pivot joystick actuator 126 including joystick 470 and ball joint coupler 150 about a center of ball 401. Although joystick actuator 126 is shown with joystick 470, any other rotational actuation means may be used for joystick actuator 126 on handle 112, such as an electronic actuator with directional buttons, a ball actuator rolled by a user's fingers, or any other actuation means known in the art.

Each articulation wires 402, 404, 406, 408 may extend outward from ball 401, from a first, distally-facing half of an exterior surface of ball 401. Articulation wires 402, 404, 406, 408 may extend longitudinally from first ball joint coupler 150 through handle 112, through second ball joint coupler 151 and proximal connector 114, through main shaft 115, first articulation shaft 120, first articulation ring 130, and second articulation shaft 122, and be fixedly coupled to second articulation ring 131. Articulation wires 402, 404, 406, 408 may be moveable proximally and distally through lumens within main shaft 115, first articulation shaft 120, first articulation ring 130, and second articulation shaft 122. Joystick actuator 126 may be configured to move second articulation shaft 122 by moving one or more of articulation wires 402, 404, 406, 408. When ball 401 is moveably coupled to handle 112, joystick 470 may be pivotable at least ninety degrees in any direction relative to neutral axis 200 of joystick 470. In some examples, joystick 470 and first ball joint coupler 150 may be spring biased towards a position in line with neutral axis 200.

FIG. 5 shows a portion of second ball joint coupler 151 removed from handle 112. Ring 501 of second ball joint coupler 151 may be fixedly coupled to a distal end portion 510 of handle 112. Ring 501 may include a central lumen 512 and an exterior surface 515 that curves towards central longitudinal axis 300 as surface 515 extends distally. In some examples, each of articulation wires 402, 404, 406, 408 of first ball joint coupler 150 may be coupled to positions on first ball joint coupler 150 evenly spaced around a perimeter portion of ball 401, and each adjacent articulation wire 402, 404, 406, 408 may be ninety degrees apart from each other adjacent actuation wire 402, 404, 406, 408. Actuation wires 402, 404, 406, 408 of first ball joint coupler 150 may extend through central lumen 512 and into main shaft 115. As shown in FIG. 5, ring 501 of second ball joint coupler 151 may include four lumens 521, 522, 523, 524 configured to receive four articulation wires 502, 504, 506, 508.

Each articulation wire 502, 504, 506, 508 may be fixedly coupled to ring 501, such that pivoting handle 112 and ring 501 relative to main shaft 115 at second ball joint coupler 151 moves at least two of articulation wires 502, 504, 506, 508. Articulation wires 502, 504, 506, 508 may be equally spaced around ring 501, and may be ninety degrees apart from each other immediately adjacent articulation wire 502, 504, 506, 508. Articulation wires 502, 504, 506, 508 may extend longitudinally from second ball joint coupler 151 through proximal connector 114, main shaft 115, and first articulation shaft 120, and may be fixedly coupled to first articulation ring 130. Articulation wires 502, 504, 506, 508 may be moveable proximally and distally through lumens within main shaft 115, and first articulation shaft 120. Rotation of handle 112 relative to main shaft 115 may move ring 501, and ring 501 may be configured to move first articulation shaft 120 by moving one or more of articulation wires 502, 504, 506, 508. Handle 112 and ring 501 may be pivoted relative to a central longitudinal axis 300 of main shaft 115. In some examples, second ball joint coupler 151 may be spring biased towards a position in line with central longitudinal axis 300 (this position is shown in FIG. 2). Actuation wires 502, 504, 506, 508 may be fixedly coupled to ring 501 at a position radially-outward from longitudinal axis 300 relative to main shaft 115, and may extend radially inward to enter main shaft 115.

FIG. 6 illustrates main shaft 115 cut across axis A (shown in FIG. 3). As shown in FIG. 6, main shaft 115 may include lumens 611, 612, 613, 614, 615, 616, 617, 618, 619 extending longitudinally through main shaft 115 from its proximal end to its distal end. Each of lumens 612, 613, 614, 615, 616, 617, 618, 619 may be configured to receive one of actuation wires 402, 404, 406, 408, 502, 504, 506, 508. Lumen 611, extending through a central portion of main shaft 115, may be configured to receive actuation wire 601. Actuation wire 601 may be coupled at its proximal end to a portion of trigger actuator 124, and may be coupled to grasper 118 at its distal end. Actuation wire 601 may be configured to move proximally when a user pulls trigger actuator 124, which may cause grasper 118 to open. An identical configuration of lumens may extend through first articulation section 120 such that FIG. 6 could be representative of cutting first articulation section 120 along axis C. Each of lumens 612, 613, 614, 615, 616, 617, 618, 619 may be evenly distributed around central longitudinal axis 300 and may be positioned the same distance radially outward from central longitudinal axis 300, and lumen 611 may extend along central longitudinal axis 300. Each of actuation wires 402, 404, 406, 408 may be positioned between actuation wires 502, 504, 506, 508 such that each of articulation wires 402, 404, 406, 408 is positioned adjacent to at least two of actuation wires 502, 504, 506, 508. In other words, wires 402, 404, 406, 408 alternate with wires 502, 504, 506, 508 about axis 300.

FIG. 7 shows a portion of distal portion 116 with second articulation shaft 122 cut along axis B of FIG. 3. As shown in FIG. 7, second articulation shaft 122 may include lumens 701, 702, 703, 704 extending longitudinally through second articulation shaft 122. Each of lumens 701, 702, 703, 704 may be configured to receive actuation wires 402, 404, 406, 408, and may longitudinally align with lumens 613, 615, 619, 617 of first articulation shaft 120 and main shaft 115. Lumen 711, extending through a central portion of second articulation shaft 122, may be configured to receive actuation wire 601. Second articulation ring 131 is removed in FIG. 7 to show actuation wire 601 extending to grasper 118. Each of articulation wires 402, 404, 406, 408 may extend entirely through second articulation shaft 122 and may be fixedly coupled to second articulation ring 131.

FIG. 20 shows a portion of distal portion 120 with first articulation shaft 120 cut along axis C of FIG. 3. As shown in FIG. 20, first articulation shaft 120 may include lumens 2001, 2002, 2003, 2004,2005,2006,2007, 2008 extending longitudinally through first articulation shaft 120. Each of lumens 2001, 2002, 2003, 2004,2005,2006,2007, 2008 may be configured to receive actuation wires 402, 404, 406, 408, 502, 504, 506, 508 and may longitudinally align with lumens 701, 702, 703, 704 of second articulation shaft 122 and 612, 613, 614, 615, 616, 617, 618, 619 main shaft 115. Lumen 2009, extending through a central portion of first articulation shaft 120, may be configured to receive actuation wire 601. Second articulation ring 130 is removed in FIG. 20 to show actuation wire 601 extending to grasper 118. Each of articulation wires 502, 504, 506, 508 may extend entirely through first articulation shaft 120 and may be fixedly coupled to second articulation ring 130.

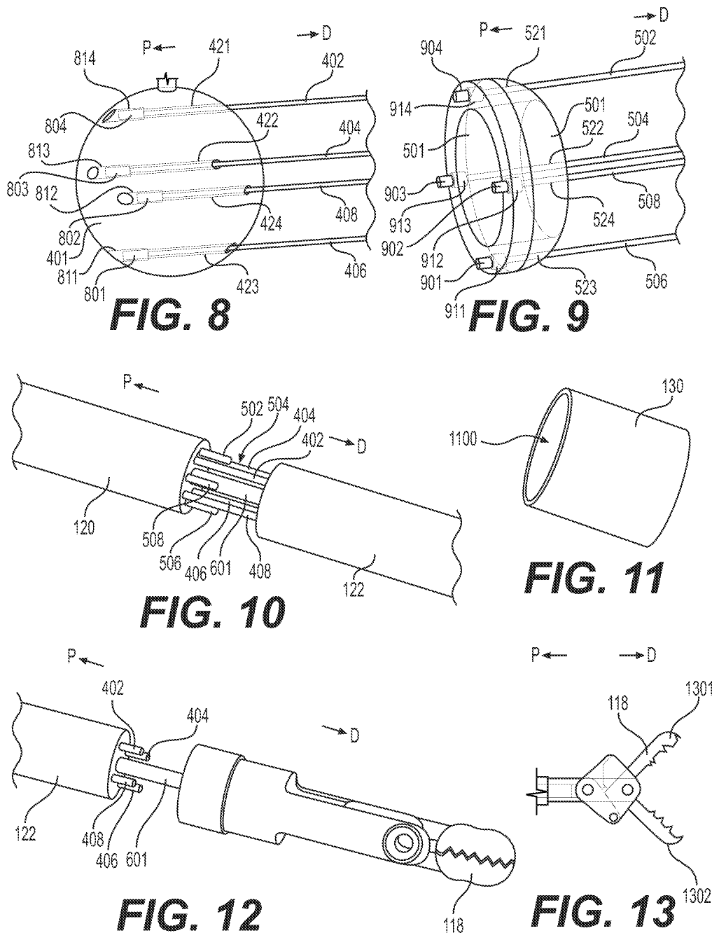

FIGS. 8 and 9 illustrate an exemplary method of coupling actuation wires 402, 404, 406, 408, 502, 504, 506, 508 to ball 401 and ring 501. Crimp sleeves 801, 802, 803, 804 may be fixedly coupled to proximal ends of articulation wires 406, 408, 404, 402, respectively. Each crimp sleeve 801, 802, 803, 804 may be positioned within a proximal end portion 811, 812, 813, 814, respectively, of each lumen 423, 424, 422, 421 in ball 401. Each proximal end portion 811, 812, 813, 814 may have a larger circumference relative to a central longitudinal axis of the lumen compared to a distal section of each lumen 423, 424, 422, 421, and may be configured to receive a crimp sleeve 801, 802, 803, 804. Each crimp sleeve 801, 802, 803, 804 may be fixedly coupled to ball 401, and thus prevent movement of each actuation wire 402, 404, 406, 408 through ball 401. Crimp sleeves 901, 902, 903, 904 may be fixedly coupled to proximal ends of articulation wires 506, 508, 504, 502, respectively. Each crimp sleeve 901, 902, 903, 904 may be positioned within a proximal end portion 911, 912, 913, 914, respectively, of each lumen 523, 524, 522, 521 in ring 501. Each proximal end portion 911, 912, 913, 914 may have a larger diameter/circumference relative to a central longitudinal axis of the lumen compared to a distal section of each lumen 523, 524, 522, 521, and may be configured to receive a crimp sleeve 901, 902, 903, 904. Each crimp sleeve 901, 902, 903, 904 may be fixedly coupled to ring 501, and thus prevent movement of each actuation wire 502, 504, 506, 508 through ring 501.

FIG. 10 illustrates a portion of distal portion 116 of medical device 110 including first articulation shaft 120 and second articulation shaft 122. First articulation ring 130 has been removed in FIG. 10 to expose articulation wires 402, 404, 406, 408, 502, 504, 506, 508, 601/wire 504 being hidden from view. Distal portions of each of articulation wires 502, 504, 506, 508 may be fixedly coupled to first articulation ring 130, and articulation wires 402, 404, 406, 408, 601 may freely move through lumen 1100 of first articulation ring 130. In some examples, first articulation ring 130 may be metal, and articulation wires 502, 504, 506, 508 may be welded to articulation ring 131. FIG. 11 illustrates an exemplary first articulation ring 130 including lumen 1100.

FIG. 12 illustrates a portion of distal portion 116 of medical device 110 including second articulation shaft 122 and grasper 118. Second articulation ring 131 has been removed in FIG. 12 to expose articulation wires 402, 404, 406, 408, 601. Distal portions of each of articulation wires 402, 404, 406, 408 may be fixedly coupled to second articulation ring 131, and articulation wire 601 may freely move through a central lumen of second articulation ring 131. In some examples, second articulation ring 131 may be metal, and articulation wires 402, 404, 406, 408 may be welded to articulation ring 131.

FIG. 13 illustrates grasper 118 in an open position with jaws 1301, 1302 separated from each other. FIG. 12 shows grasper 118 in a closed positioned with jaws 1301, 1302 clamped together, whereas FIG. 13 shows grasper 118 in an open position. In some examples, pulling trigger actuator 124 may move actuation wire 601 distally and cause grasper 118 to transition from a closed position to an open position, and releasing trigger actuator 124 may pull actuation wire 601 proximally, for example via a spring bias mechanism, and move grasper 118 to a closed position.

FIG. 14 illustrates a perspective view of distal connector 121. Distal connector 121 may be cylindrical and may include a central lumen 1401 extending the length of distal connector 121. Distal connector 121 may be configured to receive main shaft 115 of medical device 110, and may be configured to fixedly couple with proximal connector 114. For example, lumen 1401 may be configured to receive a portion of proximal connector 114 to fixedly couple proximal connector 114 to distal connector 121. A distal portion of distal connector 121 may include threads 1402, and threads 1402 may be configured to couple to working channel port 103 of endoscope 101. Distal connector 121 may provide support for a user during operation of medical device 110, and may facilitate pivoting handle 112 and rotating second ball joint coupler 151 relative to main shaft 115 and proximal coupler 114. Handle 112 may pivot relative to a center of ring 501. Distal connector 121 may also increase ergonomics for a user operating endoscope 101 using one hand and medical device 110 with the other hand by spacing handle 112 from handle 102. In operation, a user may first couple distal connector 121 to working channel port 103, and then move distal portion 116 and main shaft 115 through distal connector 121 until proximal connector 114 is inserted into lumen 1401. In an alternative embodiment, medical device 110 may not include distal connector 121 and may directly couple proximal connector 114 to working channel port 103 of endoscope 101.

FIGS. 15A, 15B, 16A, and 16B illustrate examples of the operation of medical device 110. FIGS. 15A and 15B show the proximal and distal end portions of medical device system 100, respectively. FIG. 15A shows medical device 110 positioned within working channel of endoscope 101, with distal connecter 121 coupled to endoscope 101 and handle 112 including trigger actuator 124 and joystick actuator 126. Arrows A and B in FIG. 15A illustrate movement of joystick actuator 126 in an upward and downward direction relative to handle 112, and the dotted-line versions of second articulation shaft 122 in FIG. 15B represent the resulting position of second articulation shaft 122 when joystick actuator 126 is moved upward and downward (e.g. second articulation shaft 122 is moved upward or downward). When a user moves joystick actuator 126 in the right or left directions (into and out of the page), second articulation shaft 122 may be moved in directions perpendicular to the directions shown by dotted-line versions of second articulation shaft 122 in FIG. 15B (into and out of the page).

To allow for these movements of second articulation shaft 122, articulation wires 402, 404, 406, 408 may be positioned within lumens 615, 613, 619, 617, respectively, of main shaft 115 and first articulation shaft 120 (shown in FIG. 6), and within lumens 702, 701, 703, 704, respectively, of second articulation shaft 122 (shown in FIG. 7). Alternatively, articulation wires 402, 404, 406, 408 may be positioned within lumens 619, 617, 615, 613, respectively, of main shaft 115 and first articulation shaft 120 (shown in FIG. 6), and within lumens 703, 704, 702, 701, respectively, of second articulation shaft 122 (shown in FIG. 7). In some examples, pushing joystick 470 forward (or in the distal direction labeled in FIG. 4 pivoting about the center of ball 401) may result in pulling steering wire 406 proximally, which may result in bending second articulation section 122 upward when articulation wire 406 is positioned in lumen 615 of main shaft 115/first articulation shaft 120 and lumen 702 of second articulation shaft 122. Upward direction “U”, downward direction “Do”, left direction “L”, and right direction “R” are each shown with arrows in FIGS. 6 and 7. Similarly, when joystick 470 is moved downward (in the proximal direction shown in FIG. 4, pivoting about the center of ball 401), steering wire 402 may be pulled proximally, which may result in bending second articulation section 122 downward when articulation wire 402 is positioned in lumen 619 of main shaft 115/first articulation shaft 120 and lumen 703 of second articulation shaft 122. When joystick 470 is moved in the right direction (in a direction coming out of the page in FIG. 4, pivoting about the center of ball 401), steering wire 404 may be pulled proximally, which may result in bending second articulation section 122 in the right direction when articulation wire 408 is positioned in lumen 404 of main shaft 115/first articulation shaft 120 and lumen 701 of second articulation shaft 122.

As shown in FIG. 15B, joystick actuator 126 may move second articulation shaft 122 independently of first articulation shaft 120. Articulation wires 502, 504, 506, 508 extending through first articulation shaft 120 and coupled to first articulation ring 130 may provide structural support to first articulation shaft 120 and prevent movement of first articulation shaft 120 while second articulation shaft 122 is moved via articulation wires 402, 404, 406, 408. Second articulation shaft 122 may be pivoted about first articulation ring 130 when joystick actuator 126 is moved because one or more of articulation wires 402, 404, 406, 408 may apply a force in the proximal direction on second articulation ring 131. Accordingly, second articulation shaft 122 may be moved independently of first articulation shaft 120 and main shaft 115 by actuation of joystick actuator 126. In operation, a user may move second articulation shaft 122 in any direction away from central longitudinal axis 300 by pivoting joystick actuator 126, for example with the user's thumb while operating medical device system 100. If the user wants to move main shaft 115 distally or proximally during a procedure, the user may pull handle 112 proximally or move/push the handle distally to move main shaft 115, first articulation shaft 120, and second articulation shaft 122 proximally or distally, respectively.

FIG. 16A shows medical device system 100 including medical device 110 positioned within working channel of endoscope 101, with distal connecter 121 coupled to endoscope 101. A portion of distal connector 121 is removed in FIG. 16A to show main shaft 115 extending through distal connector 121. Arrows E and F in FIG. 16A illustrate movement of handle 112 in a left direction relative to main shaft 115 and distal connector 121, with handle 112 pivoting about second ball joint coupler 151. The dotted-line versions of first articulation shaft 120 and second articulation shaft 122 represent the resulting position of first articulation shaft 120 and second articulation shaft 122 when handle 112 is moved leftward and pivoted about second ball joint coupler 151 (e.g. first and second articulation shafts 120, 122 are moved leftward). In some examples, main shaft 115 may be made of a material that is more stiff or rigid than first articulation shaft 120 and second articulation shaft 122 to prevent movement of main shaft 115 while one or more of first articulation shaft 120 and second articulation shaft 122 are moved. When a user pivots handle 112 about ball joint coupler 151 in the right or left directions, first articulation shaft 120 and second articulation shaft 122 may be moved in the right or left directions. When handle 112 is pivoted about second ball joint coupler 151 and the user does not move joystick actuator, the first articulation shaft 120 may be bent as a result of the movement of handle 112 and second articulation shaft 122 may not be bent.

Handle 112 may be pivoted about second ball joint coupler 151 in any direction, and pivoting handle 112 any direction may result in first articulation shaft 120 and second articulation shaft 122 moving in the corresponding direction relative to longitudinal axis 300 (e.g. pivoting handle 112 left results in first articulation shaft 120 and second articulation shaft 122 moving left, pivoting handle 112 downward results in first articulation shaft 120 and second articulation shaft 122 moving downward, etc.). In some examples, articulation wires 502, 504, 506, 508 may extend through lumens 614, 612, 618, 616, respectively, of main shaft 115 and first articulation section 120, which may be positioned in the upper right direction, downward right direction, downward left direction, and upward left direction, respectively. Pivoting handle 112 to the left (shown in FIG. 16A) about a center of ring 501 may pull articulation wires 506, 508 proximally, which may cause first articulation section 120 to bend to the left. Similarly, if a user pivots handle 112 upward about a center of ring 501, articulation wires 504, 506 may be pulled proximally, which may cause first articulation section 120 to bend downward.

In some examples, a user may twist their wrist in order to pivot handle 112 about second ball joint coupler 151 during operation of medical device 110. Joystick actuator 126 may be pivoted about first ball joint coupler 150 simultaneously while handle 112 is pivoted about second ball joint coupler 151, which may cause first articulation shaft 120 and second articulation shaft 122 to move and/or bend simultaneously. In some examples, if joystick actuator 126 is pivoted upward about first ball joint coupler 150 and handle 112 is simultaneously pivoted upward about second ball joint coupler 151, both first articulation shaft 120 and second articulation shaft 122 may move upward, and an upward force may be applied to distal portion 116 by two articulation wires 402, 404, 406, 408, 502, 504, 506, 508 at the same time. By providing a means to apply an upward force (or force in any other direction) utilizing two articulation wires 402, 404, 406, 408, 502, 504, 506, 508, medical device 110 provides a means to apply a force to a patient's tissue larger than the amount of force applied by a single articulation wire 402, 404, 406, 408, 502, 504, 506, 508.

As shown in FIG. 16B, second articulation shaft 122 may not itself bend when handle 112 is pivoted about second ball joint coupler 151 in order to move first articulation shaft 120. Articulation wires 402, 404, 406, 408 extending through first articulation shaft 120 and coupled to first articulation ring 130 may provide structural support to second articulation shaft 122 and prevent bending of second articulation shaft 122 while first articulation shaft 120 is moved. Second articulation shaft 122 may be pivoted about first articulation ring 130 when joystick actuator 126 is moved. Accordingly, second articulation shaft 122 may be bent/articulated independently of first articulation shaft 120 and main shaft 115 by actuation of joystick actuator 126. In operation, a user may bend/articulate second articulation shaft 122 in any direction away from central longitudinal axis 300 by pivoting joystick actuator 126, for example with the user's thumb while operating medical device system 100. At any time during operation of medical device 110, the user may actuate trigger actuator 124 in order to open or close grasper 118. Grasper 118 may be opened or closed while pivoting handle 112 about second ball joint coupler 151, while pivoting joystick actuator 126 about first ball joint coupler 150, and while both pivoting handle 112 about second ball joint coupler 151 and pivoting joystick actuator 126 about first ball joint coupler 150.

In operation of medical device system 100, a user may first insert endoscope 101 into a body of a patient and position distal portion 108 proximate to a target area within the patient's body. A user may visualize the target area using a camera 105 of endoscope 101. The user may then couple distal connector 121 to working channel port 103 of endoscope 101, and insert distal portion 116 of medical device 110 through distal connector 121 and through working channel 106 until distal portion 116 exits a distal opening of working channel 106. In other examples, a user may position medical device 110 within working channel 106 prior to insertion of endoscope 101 into the body of the patient. Once distal portion 116 exits working channel 106 and is positioned proximate to the target area, a user may move first articulation shaft 120 and second articulation shaft via pivoting joystick actuator 126 about first ball joint coupler 150 and pivoting handle 112 about second ball joint coupler 151. The user may also grab the patient's tissue by actuating trigger actuator 124 to open grasper 118, and releasing trigger actuator 124 to close grasper 118 onto tissue. In some examples, the user may grasp tissue using grasper 118, and then simultaneously pivot joystick actuator 126 upward while also pivoting handle 112 upward in order to apply the force of two articulation wires 402, 404, 406, 408, 502, 504, 506, 508 pulling upward in distal portion 116, to increase the amount of force applied to the patient's tissue compared to only utilizing a single actuation wire 402, 404, 406, 408, 502, 504, 506, 508. Once treatment of the patient is completed, the user may fully remove medical device system 100 from the patient's body.

By having two separate articulation shafts 120, 122, a user may manipulate distal section 116 of medical device 110 into positions that may not be achievable using a single articulation shaft. For example, distal section 116 may form a S-shape by bending first articulation shaft 120 to the right, and second articulation shaft 122 to the left. Distal section 116 may form a loop when first articulation shaft 120 is bent downward and second articulation shaft 122 is bent upward. By providing additional positions or shapes of distal section 116, a user may better position medical device 110 within a body of a patient compared to a conventional, single articulation shaft device.

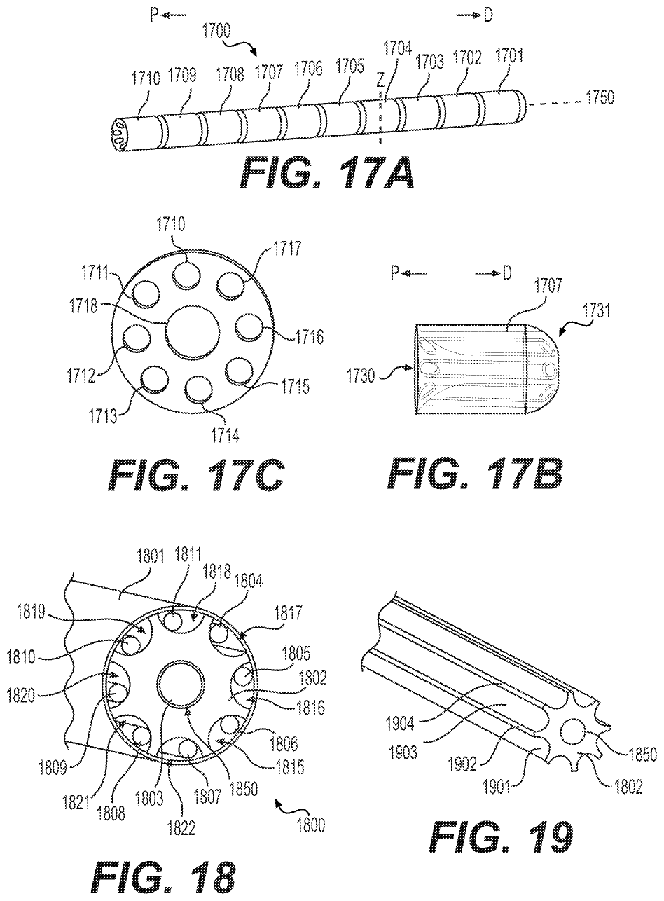

FIG. 17A illustrates a perspective view of an exemplary articulation shaft 1700. Any of first articulation shaft 120 and second articulation shaft 122 may have the structure of articulation shaft 1700. Articulation shaft 1700 may include a series of links 1701, 1702, 1703, 1704, 1705, 1706, 1707, 1708, 1709, 1710. Each link 1701, 1702, 1703, 1704, 1705, 1706, 1707, 1708, 1709, 1710 may have a cylindrical shape and may be made of any suitable material, such as metal, plastic, or any other biocompatible material. In some examples, each link 1701, 1702, 1703, 1704, 1705, 1706, 1707, 1708, 1709, 1710 may be coupled together via one or more actuation wires, and may be coupled together at their proximal and distal ends forming a chain along central longitudinal axis 1750.

FIG. 17B shows a side view of link 1707 including a curved distal end 1731 and a spherical cavity 1730 at its proximal end. Each link may be identical, and link 1707 is used for illustrative purposes Curved distal end 1731 may be ball shaped and/or hemispherical. Spherical cavity 1730 may be a concaved surface and may form a recess in link 1707. In some examples, spherical cavity 1730 may be funnel shaped and may be configured to receive a curved distal end of an immediately adjacent, more-proximal link, in this case link 1708. To allow articulation shaft 1700 to bend, each distal end 1731 of links 1701, 1702, 1703, 1704, 1705, 1706, 1708, 1709, 1710 may be moveably coupled to a spherical cavity 1730 of the link 1701, 1702, 1703, 1704, 1705, 1706, 1708, 1709, 1710 in a position immediately distal the respective link. The moveable coupling is similar to a ball-and-socket joint. FIG. 17C shows a cross-sectional front view of link 1704 cut across axis Z. As shown in FIG. 17C, each link 1701, 1702, 1703, 1704, 1705, 1706, 1708, 1709, 1710 may include nine lumens 1710, 1711, 1712, 1713, 1714, 1715, 1716, 1716, 1717, 1718 and each lumen 1710, 1711, 1712, 1713, 1714, 1715, 1716, 1716, 1717, 1718 may be configured to receive an articulation wire 402, 404, 406, 408, 502, 504, 506, 508 or actuation wire 601. Each lumen 1710, 1711, 1712, 1713, 1714, 1715, 1716, 1716, 1717, 1718 may be straight, and may be parallel with each other. Lumens 1710, 1711, 1712, 1713, 1714, 1715, 1716, 1716, 1717, 1718 are equally spaced radially about a center of link 1704. Links 1701, 1702, 1703, 1704, 1705, 1706, 1708, 1709, 1710 used in first articulation shaft 120 may include nine lumens 1710, 1711, 1712, 1713, 1714, 1715, 1716, 1716, 1717, 1718, and links used in second articulation shaft 122 may only include five lumens 1710, 1716, 1714, 1712, 1718.

FIG. 18 illustrates an embodiment of a shaft 1800 that may be used as main shaft 115 of medical device 110. Shaft 1800 may include an overtube 1801 and an inner shaft 1802. Overtube 1801 may be cylindrical and may include a central lumen extending longitudinally through overtube 1801. Inner shaft 1802 may be positioned within the lumen of overtube 1801 to form shaft 1800. Overtube 1801 may have a thickness between about 0.4 mm and about 0.7 mm, or may have any other suitable thickness. Overtube 1801 may include Pebax® such as Pebax® 72 D, fluorinated ethylene propylene (FEP), and/or any other suitable material. Lumens 1815, 1816, 1817, 1818, 1819, 1820, 1821, 1822 may be formed by a radially-inward facing surface, relative to a central longitudinal axis of shaft 1800, of overtube 1801 and a radially-outward facing surface, relative to a central longitudinal axis of shaft 1800, of inner shaft 1802. Each of lumens 1815, 1816, 1817, 1818, 1819, 1820, 1821, 1822 may be configured to receive an articulation wire 1804, 1805, 1806, 1807, 1808, 1809, 1810. A central lumen 1850 of inner shaft 1802 may extend through a central longitudinal axis of inner shaft 1802 and may be configured to receive an actuation wire 1803 or other articulation wire.

FIG. 19 shows inner shaft 1802 removed from shaft 1800. Inner shaft 1802 may extend longitudinally the entire length of shaft 1800 and may have a cross-section taken perpendicular to the central longitudinal axis of inner shaft 1802 that is the shape of an eight-pronged star. Inner shaft 1802 may include a series of curved, convex, radially outer-facing surfaces 1901, 1903. A series of prong surfaces 1902, 1904 may be formed circumferentially around the central longitudinal axis of inner shaft 1802. Each curved surface 1901, 1903 may be separated by a prong surface 1902, 1904. Each prong surface 1902, 1904 may be configured to couple to a radially-inward facing surface, relative to a central longitudinal axis of shaft 1800, of overtube 1801. Each curved surface 1901, 1903 may form part of each lumen 1815, 1816, 1817, 1818, 1819, 1820, 1821, 1822 of shaft 1800. Inner shaft 1802 may be made of PTFE or any other suitable, biocompatible material. In some examples, overtube 1801 may be glued, overmolded, and/or heat-shrinked to inner shaft 1802.

It also should also be understood that one or more aspects of any of the medical devices, systems, and methods described herein may be used for cutting, dissecting, treating, or ablating tissue in any part of the human body. For example, any of the medical devices described herein may be used in medical procedures such as for Endoscopic Submucosal Dissection (ESD), cancer treatment, and/or other procedures where removal, dissection, fulguration, and/or ablation of the type of tissue is needed.

Various aspects discussed herein may help reduce procedure time, increase tissue treatment effectiveness, reduce the risks to the subject, etc.

Although the exemplary embodiments described above have been disclosed in connection with medical devices for manipulating and resecting human tissue through the working channel of a medical device, a natural orifice, or by incision, a person skilled in the art will understand that the principles set out above can be applied to any medical device or medical method and can be implemented in different ways without departing from the scope of the disclosure as defined by the claims. In particular, constructional details, including manufacturing techniques and materials, are well within the understanding of those of skill in the art and have not been set out in any detail here. These and other modifications and variations are well within the scope of the this disclosure and can be envisioned and implemented by those of skill in the art.

Moreover, while specific exemplary embodiments may have been illustrated and described collectively herein, it should be appreciated that any subsequent arrangement designed to achieve the same or similar purpose may be substituted for the specific embodiments described and shown herein. This disclosure is intended to cover any and all subsequent adaptations or variations of various embodiments. Combinations of the above embodiments, and other embodiments not specifically described herein, will be apparent to those of skill in the art upon reviewing the description.

While principles of the disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

Other exemplary embodiments of this disclosure will be apparent to those skilled in the art from consideration of the specification and practice of the exemplary embodiments disclosed herein. It is intended that the specification and examples be considered as exemplary only, and departures in form and detail may be made without departing from the scope and spirit of this disclosure as defined by the following claims.

We claim:

1. A medical device comprising:
a main shaft extending from a proximal end to a distal end;
a first articulation shaft coupled to the distal end of the main shaft;
a second articulation shaft coupled to a distal end of the first articulation shaft;
a first articulation wire coupled proximate to a distal portion of the first articulation shaft and extending longitudinally through the main shaft and the first articulation shaft; and
a second articulation wire coupled proximate to a distal portion of the second articulation shaft and extending longitudinally through the main shaft, the first articulation shaft, and the second articulation shaft.

2. The medical device of claim 1, wherein the first articulation shaft is configured to bend independent of bending of the second articulation shaft, via movement of the first articulation wire; and
wherein the second articulation shaft is configured to bend independently of bending of the first articulation shaft, via movement of the second articulation wire.

3. The medical device of claim 1, further comprising a tool coupled to a distal end of the second articulation shaft, and an actuation wire extending through the main shaft, the first articulation shaft, and the second articulation shaft, to actuate the tool.

4. The medical device of claim 1, further comprising a first articulation ring coupled to a distal end of the first articulation shaft and coupled to a proximal end of the second articulation shaft, wherein the first articulation wire is fixedly coupled to the first articulation ring.

5. The medical device of claim 4, further comprising a second articulation ring coupled to a distal end of the second articulation shaft, wherein the second articulation wire is fixedly coupled to the second articulation ring.

6. The medical device of claim 5, further comprising:
a third articulation wire extending longitudinally through the main shaft and the first articulation shaft and fixedly coupled to the first articulation ring;
a fourth articulation wire extending longitudinally through the main shaft, the first articulation shaft, and the second articulation shaft, wherein the fourth articulation wire is fixedly coupled to the second articulation ring;
a tool coupled to a distal portion of the second articulation ring; and
an actuation wire extending longitudinally through the main shaft, the first articulation shaft, the first articulation ring, the second articulation shaft, and the second articulation ring, wherein the actuation wire is configured to actuate the tool when moved proximally or distally.

7. The medical device of claim 1, further comprising a handle coupled to the proximal end of the main shaft; wherein the handle comprises:
a first actuator rotatably coupled to the handle, wherein movement of the first actuator moves the first articulation wire; and
a second actuator coupled to the handle, the second articulation wire, and the main shaft, wherein the second actuator is rotatable relative to the main shaft, and pivoting the handle relative to the main shaft moves the second articulation wire.

8. The medical device of claim 7, wherein the handle further comprises a third actuator configured to actuate the tool.

9. The medical device of claim 7, further comprising a first connector configured to fixedly couple to a working channel port of an endoscope at a distal end of the first connector, wherein the handle is configured to rotatably couple to a proximal end of the first connector; and wherein the first connector is configured to space the handle from a handle of the endoscope.

10. The medical device of claim 8, wherein the second actuator includes a ring portion, wherein the second articulation wire is coupled to the ring portion; wherein the first articulation wire extends through a central lumen of the ring portion; and wherein the handle pivots about a center of the ring portion to move the second articulation wire.

11. The medical device of claim 7, further comprising:
third, fourth and fifth articulation wires coupled proximate to a distal portion of the first articulation shaft and extending longitudinally through the main shaft and the first articulation shaft, wherein the third, fourth and fifth articulation wires are coupled to the first actuator; and
sixth, seventh, and eighth articulation wires coupled proximate to a distal portion of the second articulation shaft and extending longitudinally through the main shaft, the first articulation shaft, and the second articulation shaft, wherein the sixth, seventh, and eighth articulation wires are coupled to the second actuator.

12. The medical device of claim 11, wherein the first actuator includes a joystick and a ball, and wherein each of the first, third, fourth, and fifth articulation wires are coupled to the ball.

13. The medical device of claim 11, wherein:
the first articulation wire is positioned within a first lumen of the main shaft and the first articulation shaft, the first lumen being directly adjacent to lumens of the main shaft and the first articulation shaft in which the second and sixth articulation wires are positioned; and
the fourth articulation wire is positioned within a second lumen of the main shaft and the first articulation shaft, the second lumen being directly adjacent to lumens of the main shaft and the first articulation shaft in which the seventh and eighth articulation wires are positioned.

14. The medical device of claim 7, wherein:
the handle is L-shaped;
the main shaft extends distally from a distal end of the handle;
the first actuator extends outward from an exterior surface of the handle and includes a joystick; and
the second actuator is positioned at a distal portion of the handle.

15. The medical device of claim 14, wherein:
pivoting the joystick is configured to bend the first articulation shaft; and
pivoting the handle relative to the main shaft is configured to bend the second articulation shaft.

16. A medical device comprising:
a main shaft extending from a proximal end to a distal end;
a first articulation shaft coupled to the distal end of the main shaft;
a second articulation shaft coupled to a distal end of the first articulation shaft;
a handle coupled to the proximal end of the main shaft, the handle comprising:
a first actuator rotatably coupled to the handle, wherein movement of the first actuator bends the first articulation shaft independent of bending the second articulation shaft; and
a second actuator coupled to the handle, wherein the second actuator is rotatable relative to the main shaft, and pivoting the handle relative to the main shaft bends the second articulation shaft independent of bending of the first articulation shaft.

17. The medical device of claim 16, wherein the handle further comprises a third actuator configured to actuate a tool at a distal portion of the medical device.

18. The medical device of claim 16, wherein the handle is a first handle, and further comprising a first connector configured to fixedly couple to a working channel port of an endoscope at a distal end of the first connector, wherein the first handle is configured to rotatably couple to a proximal end of the first connector; and wherein the first connector is configured to space the handle from a second handle of the endoscope.

19. A medical device comprising:
a main shaft extending from a proximal end to a distal end;
a first articulation shaft coupled to the distal end of the main shaft;
a second articulation shaft coupled to a distal end of the first articulation shaft;
a handle coupled to the main shaft, the handle comprising a first actuator and a second actuator;
wherein the first articulation shaft is configured to bend independent of bending of the second articulation shaft upon actuation of the first actuator; and
wherein the second articulation shaft is configured to bend independent of bending of the first articulation shaft upon actuation of the second actuator.

20. The medical device of claim 19, wherein the first articulation shaft includes a plurality of lumens, and wherein the second articulation shaft includes a plurality of lumens; and wherein the first articulation shaft includes at least one more lumen than the second articulation shaft.
